(19) United States
(12) Patent Application Publication
(10) Pub. No.: US 2017/0246204 A1
(43) Pub. Date: Aug. 31, 2017

(54) MEDICAMENT FOR TREATING A DISEASE RELATING TO A DYSFUNCTION OF THE DOPAMINERGIC SYNAPTIC TRANSMISSION

(71) Applicants: L'Air Liquide, Société Anonyme pour l'Etude et l'Explotation des Procédés Georges Claude, Paris (FR); L'Institutô de Cerveau et de la Moelle Épinière, Paris (FR)

(72) Inventors: Patrick MICHEL, Paris (FR); Jérémie LAVAUR, Paris (FR); Etienne HIRSCH, Versailles (FR); Marc LEMAIRE, Paris (FR)

(21) Appl. No.: 15/518,855

(22) PCT Filed: Sep. 7, 2015
(86) PCT No.: PCT/FR2015/052371

§ 371 Date: Apr. 13, 2017

(30) Foreign Application Priority Data
Oct. 17, 2014 (FR) 1459982

(51) Int. Cl. A61K 33/00; A61K 31/13; A61K 45/06

(52) U.S. Cl. CPC A61K 33/00; A61K 45/06; A61K 31/13

(57) ABSTRACT

The invention relates to a drug combination comprising gaseous xenon and at least one antagonist of the NMDA receptors in a liquid or solid form for treating a disease caused by a dysfunction of the dopaminergic synaptic transmission in a human patient. The antagonist of the NMDA receptors is preferably selected from memantine, nitromemantine, amantadine and ifenprodil. The invention allows the normal function of the dopaminergic synaptic transmission of diseased neurons with an altered function to be reestablished.

### CROSS REFERENCE TO RELATED APPLICATIONS

This application is a 371 of International PCT Application PCT/FR2015/052371 filed Sep. 7, 2015, which claims priority to French Patent Application No. 1459982 filed Oct. 17, 2014, the entire contents of which are incorporated herein by reference.

### BACKGROUND

The invention relates to the use of xenon gas as an inhalable drug, used in combination with an N-methyl-D-aspartate (NMDA) glutamate receptor antagonist for treating, that is to say for curing, slowing down the progression of, attenuating or minimizing, a central nervous system disease resulting from a dysfunction of dopaminergic synaptic transmission.

NMDA receptors/channels are molecular entities of the neuronal cell plasma membrane. These receptors are the target of glutamate molecules released into the synaptic and extrasynaptic space, glutamate being an excitatory neural transmitter which provides communication from one nerve cell to another.

Dopaminergic transmission is impaired in a certain number of central nervous system pathologies or pathological conditions, in particular Parkinson's disease, dyskinesia, schizophrenia, restless legs syndrome, Tourette's syndrome, addictions, major depression and attention deficit disorders, with or without hyperactivity.

Memantine, nitromemantine, amantadine and ifenprodil are compounds which have an antagonist action with respect to the NMDA receptor carried by neurons, but these molecules are also capable of producing a neurotrophic effect via a mechanism involving astrocyte-type glial cells (Toyomoto et al., Neurosci Letters, 2005; Wu et al., Neuropsychopharmacology, 2009; Ossola et al., Neuropharmacology, 2009). This then results in an enhancement of nerve transmission between neural cells.

However, this positive effect of NMDA receptor antagonists, in particular memantine, is limited because these molecules are not without adverse effects, such as confusion, dizziness, drowsiness, headaches, insomnia, agitation, hallucinations, vomiting, anxiety, etc., which counteract their positive effect.

The problem is therefore that of providing a drug which makes it possible to treat, slow down or minimize a central nervous system disease caused by, or resulting from, a dysfunction of dopaminergic synaptic transmission in the case of certain pathologies or pathological conditions, in particular Parkinson's disease, dyskinesia, schizophrenia, restless legs syndrome, Tourette's syndrome, a major depression and attention deficit disorders with or without hyperactivity (Oades, Prog Brain Res, 2008; Beaulieu and Gainetdinov, Pharmacol Rev, 2011; Michel et al., Faseb J, 2013; Ferini-Strambi and Marelli, Expert Opin Pharmacother, 2014), while at the same time limiting the abovementioned adverse effects associated with the use of memantine.

### SUMMARY

The solution according to the present invention is a drug containing xenon gas for use in combination with at least one NMDA receptor antagonist in liquid or solid form, for treating a central nervous system disease resulting from a dysfunction of dopaminergic synaptic transmission in a human patient.

In the context of the invention, the term “treating” is understood in the broad sense and encompasses not only “curing”, but also “slowing down the progression of”, “attenuating” or “minimizing” the disease. The treatment is thus total or partial.

In other words, the invention relates to a drug combination comprising xenon gas and at least one NMDA receptor antagonist in liquid or solid form for treating a disease caused by a dysfunction of dopaminergic synaptic transmission in a human patient.

Indeed, in the context of the present invention, it has been demonstrated that the combination of xenon and an NMDA receptor antagonist such as memantine, results in a synergistic action of these compounds and that such a combination can constitute a promising treatment for central nervous system diseases resulting from a dysfunction of dopaminergic synaptic transmission, in particular Parkinson's disease, dyskinesia, schizophrenia, restless legs syndrome, Tourette's syndrome, a major depression and attention deficit disorders with or without hyperactivity.

Such a combination is based in particular on the modes of action of these compounds.

Thus, xenon has excitatory glutamatergic signaling pathway-inhibiting properties (Dinse et al., Br J Anaesth, 2005), by its antagonistic action on NMDA receptors, but also on α-amino-3-hydroxy-5-methylisoazole-4-propionate (AM PA) receptors, and also on kainate receptors, which make up ionotropic glutamate receptors.

Consequently, the combination of xenon with an NMDA receptor antagonist compound, in particular memantine, nitromemantine, amantadine and ifenprodil, results in a synergy of action, without the risk of increasing the adverse effects of the NMDA receptor antagonist, since such a combination makes it possible to use lower doses of the antagonist used.

In other words, xenon makes it possible to reinforce the beneficial effects of the NMDA receptor antagonist, in particular memantine, owing to a synergistic effect, but without causing adverse effects, such as confusion, dizziness, drowsiness, headaches, insomnia, agitation, hallucinations, vomiting, anxiety, etc.

Generally, such a combination makes it possible to reestablish the synaptic transmission of “diseased” dopaminergic neurons of which the function is impaired.

As appropriate, the gaseous drug or drug combination according to the invention may comprise one or more of the following features:
the xenon is in a form that can be administered by inhalation, that is to say that is intended to be and capable of being inhaled by the patient;
the NMDA receptor antagonist is in solid form, in particular in tablet or gel capsule form;
the NMDA receptor antagonist is chosen from memantine, nitromemantine, amantadine and ifenprodil, or any other analogous compound;
the disease caused by a dysfunction of dopaminergic synaptic transmission is chosen from Parkinson's disease, dyskinesia, schizophrenia, restless legs syndrome, Tourette's syndrome, addictive behaviors, severe depression and attention deficit disorders with or without hyperactivity;
the disease results from, or is caused by, a dysfunction of dopaminergic synaptic transmission caused by, or resulting from, attrition of the cell body, a decrease in neurite arborization or a reduction in synaptic function;
the daily dose of NMDA receptor antagonist administered to the patient is less than or equal to 20 mg/day;
the xenon gas is administered to the patient before, concomitantly with or after administration of the NMDA receptor antagonist, preferably after administration of the NMDA receptor antagonist;
the xenon is mixed with oxygen, preferably with at least 21% by volume of oxygen;
the patient is a man or a woman, whether said patient is an adult or a child;
the drug contains an effective amount of xenon;
the drug contains a non-anesthetic amount of xenon, i.e. a subanesthetic amount of xenon;
the proportion of xenon is between 10% and 80% by volume;
the proportion of xenon is between 15% and 80% by volume;
the proportion of xenon is at least 20% by volume;
the proportion of xenon is at most 75% by volume;
the proportion of xenon is at most 60% by volume;
   the proportion of xenon is between 20% and 50% by volume;
   the proportion of xenon is between 20% and 40% by volume;
   the xenon is mixed with oxygen just before or at the time of its inhalation by the patient or is in the form of a “ready-to-use” gas mixture as a premixture with oxygen, and optionally contains another gaseous compound, for example nitrogen;
a mixture consisting of xenon and oxygen is used;
   a mixture consisting of xenon, nitrogen and oxygen is used;
   the gas mixture is administered to the patient one or more times per day;
   the gas mixture is administered to the patient for an inhalation time of a few minutes to a few hours, typically between 15 minutes and 6 hours, preferentially less than 4 hours;
   the duration, dose regimen and frequency of administration depend on the progression of the neurological condition of the patient under consideration and will preferentially be set by the physician or care staff according to the neurological condition of the patient under consideration;
   the xenon or the gas mixture is packaged in a gas cylinder having a volume (water equivalent) ranging up to 50 liters, typically of about from 0.5 to 15 liters and/or at a pressure of less than or equal to 350 bar absolute, typically a pressure of between 2 and 300 bar;
   the xenon or the gas mixture is packaged in a gas cylinder equipped with a valve or a pressure regulator that is integrated, making it possible to control the flow rate and optionally the pressure of the gas delivered;
   the xenon or the gas mixture is packaged in a gas cylinder made of steel, aluminum or composite materials;
   during the treatment, the xenon or the xenon-based gas mixture is administered to the patient by inhalation by means of a face mask or nasal mask or of nasal goggles or by means of any other system for administration of an inhalable gas.

The invention also relates to the use of xenon gas and of at least one NMDA receptor antagonist in liquid or solid form, for producing a drug that can be used for treating a disease caused by a dysfunction of dopaminergic synaptic transmission in a human patient.

As appropriate, the use according to the invention may comprise one or more of the following technical features:
the gaseous drug or the drug combination comprises one or more of the features described above;
the NMDA receptor antagonist is chosen from memantine, nitromemantine, amantadine and ifenprodil;
the disease caused by a dysfunction of dopaminergic synaptic transmission is chosen from Parkinson's disease, dyskinesia, schizophrenia, restless legs syndrome, Tourette's syndrome, addictive behaviors, severe depression and attention deficit disorders with or without hyperactivity;
the disease results from, or is caused by, a dysfunction of dopaminergic synaptic transmission resulting from attrition of the cell body, a decrease in neurite arborization or a reduction in synaptic function.

According to another aspect, the invention also relates to a method of therapeutic treatment for treating or slowing down a central nervous system disease resulting from a dysfunction of dopaminergic synaptic transmission, where in said method:

i) a human patient suffering from a central nervous system disease resulting from a dysfunction of dopaminergic synaptic transmission is identified,

ii) a gaseous drug containing xenon is administered to said patient by inhalation, and

iii) at least one NMDA receptor antagonist in liquid or solid form is administered to said patient so as to treat said central nervous system disease.

According to this method of therapeutic treatment, the combination of xenon and the NMDA receptor antagonist results in a combined action of these compounds making it possible to restore normal synaptic function, thus resulting in a treatment, in particular at least a slowing down of the disease.

In particular, such a disease is chosen from Parkinson's disease, dyskinesia, schizophrenia, restless legs syndrome, Tourette's syndrome, addiction, major depression or attention deficit disorders with or without hyperactivity in said patient.

Preferably, in step i):
the human patient is a man or a woman, whether said patient is an adult or a child;
the patient is identified by a physician or the like;
the patient is identified by technical screening examination;
the dopaminergic neuron synaptic function abnormality is capable of causing a neurological dysfunction.

Preferably, in step ii):
at least one NMDA receptor antagonist in solid form is administered to said patient;
at least one NMDA receptor antagonist is preferably administered enterally, i.e. orally;
at least one NMDA receptor antagonist in tablet or gel capsule form is administered to said patient;
memantine or a memantine-derived compound is administered to the patient as NMDA receptor antagonist;
nitromemantine is administered to the patient as NMDA receptor antagonist;
amantadine or ifenprodil is administered to the patient as NMDA receptor antagonist;
a daily dose of NMDA receptor antagonist of less than or equal to 20 mg/day is administered to the patient;
at least one NMDA receptor antagonist is administered to said patient before, concomitantly with or after inhalation of xenon by the patient.

Preferably, in step iii):
the duration, the dosage regimen and the frequency of administration of the xenon are chosen and/or set according to the progression of the neurological condition of the patient under consideration;
an effective amount of xenon is administered;
a non-anesthetic amount of xenon is administered;
   from 10% to 75% by volume of xenon, preferably between 20% and 50% by volume of xenon, is administered;
   the xenon is mixed with oxygen before or at the time of its inhalation by the patient, preferably with at least 21% by volume of oxygen;
   a ready-to-use gas mixture consisting of xenon and oxygen (binary mixture) is administered;
   a ready-to-use gas mixture consisting of xenon, oxygen and nitrogen (ternary mixture) is administered;
   the xenon gas is administered to the patient one or more times per day;
   the xenon gas is administered to the patient for an inhalation time of a few minutes to a few hours, typically between 15 minutes and 6 hours, preferentially less than 4 hours;
   the xenon gas is administered by means of a face mask or nasal mask or of nasal goggles or by means of any other system or device for administration of gas to a patient.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:
FIG. 1 illustrates tritiated dopamine reuptake, considered to be an index of synaptic function and of differentiation of dopaminergic neurons, is measured on living cells at D14.
FIG. 2 illustrates the neurite length per TH+ neuron, which is a measurement of the potential of dopaminergic neurons to be able to form synaptic connections with other neurons, is carried out on cultures fixed on D14.

### DESCRIPTION OF PREFERRED EMBODIMENTS

In order to demonstrate the synergistic effect of the combination of xenon and an NMDA receptor antagonist according to the present invention, a cell model was set up, in which the dopaminergic synaptic transmission dysfunction is spontaneous.

This dysfunction is characterized by attrition of the cell body, a decrease in neurite arborization and also a reduction in synaptic function, under the culture conditions used (Wu et al., Neuropsychopharmacology, 2009).

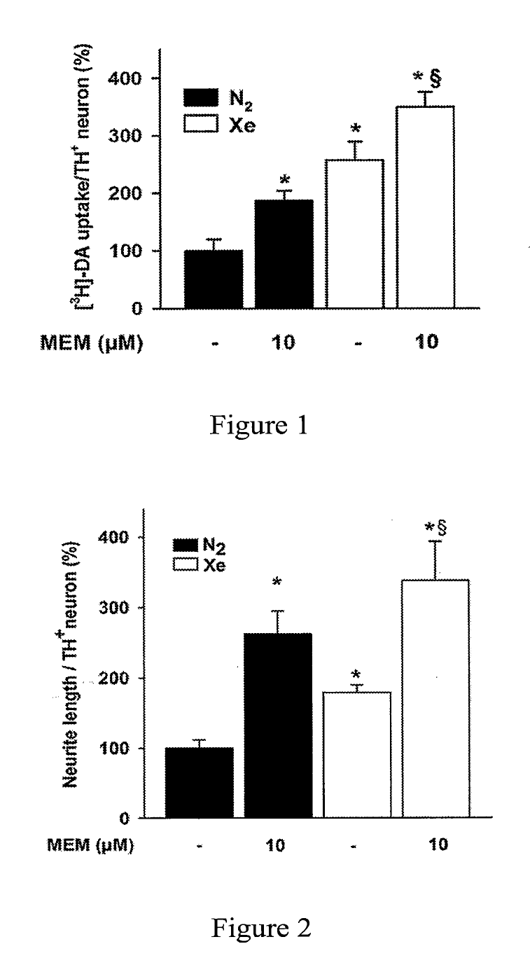

The technique implemented is described below and the results obtained are given in Tables 1 and 2 and illustrated by FIGS. 1 and 2 appended hereto, showing the synergistic effects of xenon combined with memantine in a cell model mimicking neuronal attrition in connection with a dysfunction of dopaminergic synaptic transmission.

### EXAMPLES

### Protocol for Obtaining Primary Cultures of Mesencephalon

Cultures are prepared from mesencephalon of rat embryos, taking from female Wistar rats, on day 15.5 of gestation.

The process for obtaining the mesencephalon cultures comprises the obtaining of a homogeneous cell suspension by mechanical dissociation, that is to say non-enzymatic dissociation, of the embryonic tissue, using Leibovitz's L15 medium (Sigma Aldrich).

Aliquots of this suspension are added to 48-well Nunc plates, which have been precoated with a thin layer of polyethyleneimine (1 mg/ml, borate buffer, pH 8.3) so as to allow the adhesion of neuronal cells (Toulorge et al., Faseb J, 2011).

The seeding density is between approximately 80 000 and 100 000 cells/cm².

The mesencephalon cultures are maintained in Minimum Essential culture Medium (MEM), containing 1 g/l of glucose, 2 mM of L-glutamine, 1 mM of sodium pyruvate, non-essential amino acids and a penicillin/streptomycin cocktail. For the first week of culture, this medium is supplemented with 10% of fetal calf serum and 10% of horse serum. Starting from the second week, the serum concentrations are reduced to 2% (Gao et al., J Neurosci, 2002).

Up until the moment when the effects of the gases of interest are evaluated, the cultures are placed in a conventional enclosure thermostated at 37° C., in which the CO₂ is maintained at 5% by volume and wherein the atmosphere is saturated with water.

The dopaminergic neuron dysfunction process which spontaneously sets in results from the neuronal attrition and the reduction in neurite arborization.

### Pharmacological Treatments of the Cultures

An NMDA receptor blocker, namely memantine, is added to the cultures just before they are placed under a controlled gas atmosphere and the treatment is prolonged until the cultures are fixed.

### Maintenance of the Cultures Under a Controlled Gas Atmosphere

Once the pharmacological treatments have been carried out, the multiwell dishes containing the cells in culture and the dish used to humidify the internal compartment of the chamber are placed on a metal base which receives the Plexiglas incubation chamber. The base and the Plexiglas chamber are butt-joined together by a screw.

A gas mixture of interest comprising (% by volume): 20% of O₂, 5% of CO₂ and 75% of test gas is then injected into the incubation chamber, with open inlet and outlet valves, while at the same time controlling the output flow rate by means of a flow meter. The test gases are nitrogen (N₂) and xenon (Xe).

The reference output flow rate, set for air at 10 liters/min, is corrected according to the density of the mixture used. When the CO₂ measurement reaches 5% at the outlet, the injection of the gas mixture is stopped and the chamber is made totally airtight by closing the inlet and outlet valves. The exposure chamber is then placed in an enclosure at 37° C. for the 7 days of the experimental protocol.

### Immunodetection of the Tyrosine Hydroxylase (TH) Protein and Cell Counts

After breaking the airtightness by opening the inlet and outlet valves and unscrewing the chamber from its base, the cultures are fixed with 4% formaldehyde in PBS for 12 min and then incubated at 4° C. with an anti-TH monoclonal antibody (ImmunoStar; dilution 1:5000) for 2 days, so as to reveal the presence of the dopaminergic neurons.

The revelation by this antibody is achieved with an anti-mouse secondary antibody coupled to Alexa Fluor 555 (Life Technologies; dilution 1:300 in PBS).

The image acquisition and the counting of the dopaminergic neurons (TH⁺) are carried out with an automated imager of Arrayscan XTI type and the HCS Studio image analysis software (ThermoFischer Scientific). This analysis makes it possible to have an estimation of the number of TH⁺ neurons/culture well.

### Measurement of the Neurites Borne by the Dopaminergic Neurons

The cultures used for counting the dopaminergic neurons are also used for the measurement of the length of the neurites borne by the dopaminergic TH⁺ neurons.

This parameter is evaluated with the same HCS Studio software.

### Measurement of Tritiated Dopamine Reuptake

The dopamine reuptake measurement is carried out using tritiated dopamine (30-60 Ci/mmol, PerkinElmer), according to a protocol previously described by Toulorge et al. (Faseb J, 2011). The reuptake measurement is carried out in cultures produced at the same time and under the same conditions as those used for the cell counting studies. For each culture well, the level of tritiated dopamine reuptake is related to the mean estimated value of the number of TH⁺ dopaminergic neurons in each treatment condition.

The results obtained in this cell model of dopaminergic synaptic transmission dysfunction are summarized in the following Tables 1 and 2 and are represented in FIGS. 1 and 2, appended hereto.

TABLE 1
tritiated dopamine reuptake by dopaminergic neuron
Cultures of mesencephalon at D 14

| Treatments | Gas mixture | Dopamine |
|---|---|---|
| D 7-D 14 | (20% O₂ + 5% CO₂ + 75% | reuptake/TH⁺ |
| (7 days) | gas tested); % by volume | neuron |
| Control group | N₂ | − |
| Memantine | N₂ | + |
| (10 μM) group | | |
| Xenon alone group | Xe | ++ |
| Xenon + memantine | Xe | +++ |
| (10 μM) group | | |

TABLE 2
neurite length/dopaminergic neuron
Cultures of mesencephalon at D 14

| Treatments | Gas mixture | |
|---|---|---|
| D 7-D 14 | (20% O₂ + 5% CO₂ + 75% | neurites/TH⁺ |
| (7 days) | gas tested); % by volume | neuron |
| Control group | N₂ | − |
| Memantine | N₂ | ++ |
| (10 μM) group | | |
| Xenon alone group | Xe | + |
| Xenon + memantine | Xe | +++ |
| (10 μM) group | | |

In the two tables above, a favorable response, synonymous with an increase in dopamine reuptake per TH⁺ neuron (Table 1) or with an increase in neurite length per TH⁺ neuron (Table 2), in the presence of the treatments of interest, is denoted by a “+”, “++” or “+++” sign, depending on the size of this response.

Conversely, an absence of response is represented by a “—” sign.

In the light of the results obtained, illustrated in the figures appended hereto, it is noted that the combination of xenon and memantine, which is an NMDA receptor antagonist, results in a synergistic effect that is significantly greater than that produced by each molecule, taken separately, whether the parameter measured is tritiated dopamine reuptake/TH⁺ neuron or neurite length/TH⁺ neuron.

Memantine exerts a significant effect in this cell model and with respect to the two parameters studied, when it is applied alone at 10 μM, under an atmosphere containing 75% nitrogen. Xenon also has an activity that is likewise significant when it replaces nitrogen. Furthermore, xenon potentiates the effects obtained in the presence of memantine.

The results illustrated in FIGS. 1 and 2 reveal the synergistic effects of xenon and memantine in the cell model of dopaminergic synaptic dysfunction.

These results were obtained on rat mesencephalon cultures which were placed, from day 7 of culture up to day 14, under an atmosphere containing 75% nitrogen (N₂ 75) or 75% xenon (Xe 75), in the presence or absence of memantine (MEM), tested at 10 μM.

In FIG. 1, the tritiated dopamine reuptake, considered to be an index of synaptic function and of differentiation of dopaminergic neurons, is measured on living cells at D14.

The number of TH⁺ dopaminergic neurons is estimated on sister cultures, which are produced from one and the same starting cell suspension and are fixed on D14.

The levels of dopamine reuptake per TH⁺ neuron are expressed as % (±SEM) of nontreated cultures, cultured under an atmosphere containing 75% nitrogen (control condition).

Thus, the statistical study carried out by means of a Kruskal-Wallis ANOVA (ANalysis Of VAriance) by ranks, followed by a Student-Newman-Keuls test (n=9 for each experiment point), demonstrates that:
xenon alone when it replaces nitrogen, memantine alone in nitrogen and memantine in xenon produce an effect that is significantly greater than that observed under the nitrogen alone condition (*p<0.05, increased compared with control cultures at D14 under 75% nitrogen);
xenon when it is combined with memantine causes a synergistic effect and results in a result that is significantly greater than that observed under the xenon alone or memantine under nitrogen conditions (^{§}p<0.05, increased at D14 compared with cultures exposed to xenon alone or to memantine under an atmosphere containing nitrogen).

In FIG. 2, the neurite length per TH⁺neuron, which is a measurement of the potential of dopaminergic neurons to be able to form synaptic connections with other neurons, is carried out on cultures fixed on D14.

The results are expressed as % (±SEM) of nontreated cultures, maintained in a control atmosphere containing 75% nitrogen (control condition).

Thus, the statistical study carried out by means of a Kruskal-Wallis ANOVA (ANalysis Of VAriance) by ranks, followed by a Student-Newman-Keuls test (n=9 for each experimental point), demonstrates that:
xenon alone when it replaces nitrogen, memantine alone in nitrogen and memantine in xenon exert an effect that is significantly greater than that observed under the nitrogen alone control condition (*p<0.05, increased compared with control cultures at D14 under 75% nitrogen);
xenon when it is combined with memantine exerts an effect that is significantly greater than that observed under the xenon alone or memantine under nitrogen conditions (^{§}p<0.05, increased at D14 compared with cultures exposed to xenon alone or to memantine in an atmosphere containing nitrogen).

The above tests were carried out with memantine as NMDA receptor antagonist. However, these similar results would be obtained with compounds of the same type, analogs or the like, such as nitromemantine, amantadine or ifenprodil.

Xenon, when it is combined with an NMDA receptor antagonist, such as memantine, nitromemantine, amantadine or ifenprodil, thus results in a synergistic effect in the treatment, in particular the slowing down of the progression, of diseases associated with a dysfunction of dopaminergic synaptic transmission, such as Parkinson's disease, dyskinesia, schizophrenia, restless legs syndrome, Tourette's syndrome, addiction, depression when it is major depression, and attention deficit disorders with or without hyperactivity.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A method for treating a disease caused by a dysfunction of dopaminergic synaptic transmission in a human patient comprising the step of administering a drug combination comprising xenon gas and at least one NMDA receptor antagonist in liquid or solid form to thereby treat the disease caused by the dysfunction of dopaminergic synaptic transmission in the human patient.

2. The method of claim 1, wherein, in the drug combination, the proportion of xenon is between 10% and 80% by volume.

3. The method of claim 1, wherein the NMDA receptor antagonist is in solid form.

4. The method of claim 1, wherein the NMDA receptor antagonist is chosen from memantine, nitromemantine, amantadine and ifenprodil.

5. The method of claim 1, wherein the disease caused by a dysfunction of dopaminergic synaptic transmission is chosen from Parkinson's disease, dyskinesia, schizophrenia, restless legs syndrome, Tourette's syndrome, addictive behaviors, and/or severe depression and attention deficit disorders with or without hyperactivity.

6. The method of claim 1, wherein the disease results from, or is caused by, a dysfunction of dopaminergic synaptic transmission resulting from attrition of the cell body, a decrease in neurite arborization or a reduction in synaptic function.

7. The method of claim 1, wherein xenon gas is mixed with oxygen so that the xenon gas is at least 21% by volume of oxygen.

8. The drug combination method of claim 1, wherein the xenon is mixed with oxygen or with oxygen and nitrogen.

9. The method of claim 1, wherein the proportion of xenon in the xenon gas is at least 20% by volume.

10. The method of claim 1, wherein the proportion of xenon in the xenon gas is at most 75% by volume.

11. The method of claim 1, wherein the proportion of xenon in the xenon gas is at most 60% by volume.

12.-15. (canceled)
